(19) United States
(12) Patent Application Publication
(10) Pub. No.: US 2014/0107464 A1
(43) Pub. Date: Apr. 17, 2014

(54) Method for Locating a Brain Activity

(71) Applicant: Commissariat A L'Energie Atomique Et Aux Energies Alternatives, Paris (FR)

(72) Inventors: Tetiana Aksenova, Saint-Egreve (FR); Etienne Labyt, St. Martin Le Vinoux (FR); Ales Mishchenko, St. Petersbourg (RU)

(21) Appl. No.: 13/928,632

(22) Filed: Jun. 27, 2013

(30) Foreign Application Priority Data
Jun. 29, 2012 (FR) 12 56292

(51) Int. Cl. A61B 5/05

(52) U.S. Cl. CPC A61B 5/05
USPC 600/409

(57) ABSTRACT

Method for locating a brain activity, comprising the steps consisting in:
a) acquiring data indicative of sensory stimuli addressed to, or deliberate actions performed or imagined by, a subject;
b) by means of a plurality of sensors, acquiring signals representative of an activity, associated with said stimuli or deliberate actions, of respective regions of the brain of said subject; and
c) for each said sensor, quantifying a correlation that exists between said data indicative of sensory stimuli or of deliberate actions and the signals acquired;
   characterized in that, for each said sensor, said correlation is established between a scalar variable indicative of a said sensory stimulus or of a said deliberate action and a multidimensional variable representative of signals that are associated therewith.

Application of this method to the determination of optimum locations for brain activity sensors for direct neural control.

The invention relates to a method for locating brain activity in a subject, notably by magneto-encephalography. The invention applies in particular to the field of direct neural control.

Direct neural control (or “brain-computer interface”, BCI) makes it possible to establish a communication between a user and a machine (typically a computer) through neural signals deriving from the brain activity of a subject without making use of the muscular pathway, which constitutes a real hope for people suffering from serious paralyses.

The non-intrusive direct neural control systems use, more often than not, electroencephalography (EEG) as the method for acquiring brain activity. Thus, a certain number of electrodes are fixed on the surface of the cranium in order to measure therein an electrical activity reflecting the brain activity of the subject. Other techniques, more efficient but also more intrusive, exploit electrocorticographic (ECoG) signals, taken on the surface of the cortex, even signals taken by deep electrodes.

Magnetoencephalography (MEG) is a non-intrusive technique, the use of which in direct neural control is conceptually interesting, because the magnetic signals suffer little or no distortion when they are propagated through the cranium. On this subject reference can be made to the paper by J. Mellinger et al. “An MEG-based Brain-Computer Interface (BCI)”, Neuroimage 36(3), 581-593 (1 Jul. 2007).

The main drawback with this technique, which in practice limits it to experimental applications, is the insufficient miniaturization of the magneto-encephalographic sensors.

Whatever the method used for acquiring the brain activity, the basic principle of direct neural control generally consists in associating one or more mental tasks (actions imagined by the subject) with one or more actions performed by an effector. For example, the imagination of the movement of the right hand can be associated with the displacement to the right of a cursor.

The inclusion of the spatial information conveyed by the neural signals is important in achieving this association. In practice, performing different mental tasks activates different regions of the brain, or the same regions but in a different manner. To maximize the preservation of this spatial information, a large number of sensors (up to a hundred or so) are used in most cases. This approach presents a number of drawbacks: a nuisance to the user, long preparation time, high computational cost. Furthermore, certain types of treatment show limitations when the number of sensors increases (for example, overlearning effects are observed).

Thus, techniques have been developed to determine the optimal placements, on the cranium or on the surface of the cortex of a subject, where as limited a number of sensors as possible can be located. For example, the paper by A. Barachant, T. Aksenova and S. Bonnet, “Filtrage spatial robuste à partir d' un sous-ensemble optimal d' électrodes en BCI EEG” [“Robust spatial filtering from an optimum subset of electrodes in BCI EEG”], GRETSI 2009, 8-11 Sep. 2009, describes an ascending selection method (that is to say in which an optimal set of sensors is progressively constructed), based on a criterion of multiple correlation of the log-variance of the EEG signals after frequency filtering.

The paper by F. Tadel et al. “Brainstorm: A User-Friendly Application for MEG/EEG Analysis”, Computational Intelligence and Neuroscience, Vol. 2011, Article ID 879716, (2011) describes software for analyzing electroencephalographic and magnetoencephalographic signals that notably enables them to be represented in the form of time-frequency maps. This technique makes only incomplete use of the information supplied by the magnetoencephalographic sensors. In practice, such a sensor generally comprises three individual sensors or more, and notably:
a magnetometer which measures the intensity of the magnetic field at a point; and
two planar gradiometers, mutually perpendicular, which measure two components of the gradient of said magnetic field.

However, for the determination of the brain regions that are activated in a BCI experience, only a single signal is generally used that is representative of the modulus of the gradient of the magnetic field—without taking into account its direction or the intensity of the field.

The invention aims to provide a method for locating brain activity in a subject that allows for a better use of the information acquired by the various sensors.

According to the invention, such an aim is achieved by a method for locating a brain activity, comprising the steps consisting in:

a) acquiring data indicative of sensory stimuli addressed to, or deliberate actions performed or imagined by, a subject;

b) by means of a plurality of sensors, acquiring signals representative of an activity, associated with said stimuli or deliberate actions, of respective regions of the brain of said subject; and

c) for each said sensor, quantifying a correlation that exists between said data indicative of sensory stimuli or of deliberate actions and the signals acquired;

characterized in that each said sensor acquires N≧2 signals that are representative of N distinct physical quantities, measured for one and the same region of the brain, and in that said correlation is established between a variable, and notably a scalar variable, indicative of a said sensory stimulus or of a said deliberate action and a variable of N or at least N dimensions representative of said signals.

In other words, N distinct signals are acquired, N≧2, in one and the same region of the brain, and a correlation is established between a variable representative of an action imagined or performed by a subject, and a variable comprising at least N components, each component depending on one of the N signals measured.

In particular, a correlation is established between a variable representative of an action imagined or performed by a subject, and a variable comprising at least N components, each component depending on only one of the N signals measured.

Said variable comprising at least N components can notably comprise values of the N signals measured in one and the same region of the brain, by a sensor or a group of sensors, these signals being offset in time relative to the variable representative of the action imagined or performed by the subject.

The same region of the brain should be understood to mean a spatial area inscribed in a circle of diameter less than 1 cm, even less than 0.5 cm, even less than 0.1 cm.

Advantageously, said correlation that exists between said data indicative of sensory stimuli or of deliberate actions and the signals acquired by each said sensor can be quantified by means of a determination coefficient.

By way of comparison, notably when using magnetoencephalographic measurement means, the methods known from the prior art and making use of the time-frequency maps are intrinsically limited to taking into account signals of scalar type (for example: measurements of the modulus of the gradient of the magnetic field or of the intensity of the magnetic field).

In particular, said sensors can be magnetoencephalographic sensors. In this case, each said magnetoencephalographic sensor can acquire at least one signal representative of a magnetic field intensity and one signal representative of a component of a gradient of said magnetic field; as a variant or in complement, each said magnetoencephalographic sensor can acquire at least two signals representative of two components of a gradient of said magnetic field. Thus, the notion of sensor must be taken in a wide sense; within the meaning of the invention, a sensor is capable of acquiring a plurality of signals at one and the same point or in one and the same spatial area.

Said variable with N dimensions can be obtained by selecting at least one spectral component of each of said signals, acquired over a time window associated with a said sensory stimulus or deliberate action.

According to another embodiment of the invention, said multidimensional variable can be obtained by selecting a plurality of spectral components of a signal acquired over a time window associated with a said sensory stimulus or deliberate action.

These embodiments can be combined: thus, said multidimensional variable can comprise a plurality of spectral components of a plurality of signals representative of respective physical quantities.

The method can also comprise a visualization step in which values indicative of said correlation, determined for each said sensor, are projected onto a three-dimensional model of a cortical surface, and an interpolation of said values between different points of a mesh of said surface is performed.

Another subject of the invention is a method for locating brain activity sensors for direct neural control comprising:
a step of locating a brain activity, implemented by a method as described above; and
a step of determining optimum locations of said brain activity sensors according to the results of said step of locating a brain activity.

Other features, details and advantages of the invention will become apparent on reading the description given with reference to the appended drawings, given by way of example and which represent, respectively:

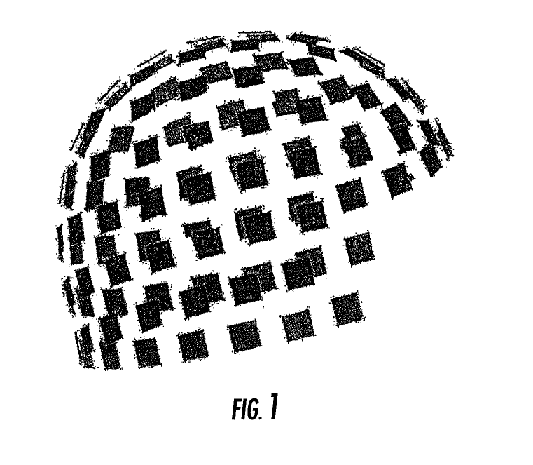

FIG. 1, a headset for magnetoencephalography used to implement a method according to the invention;

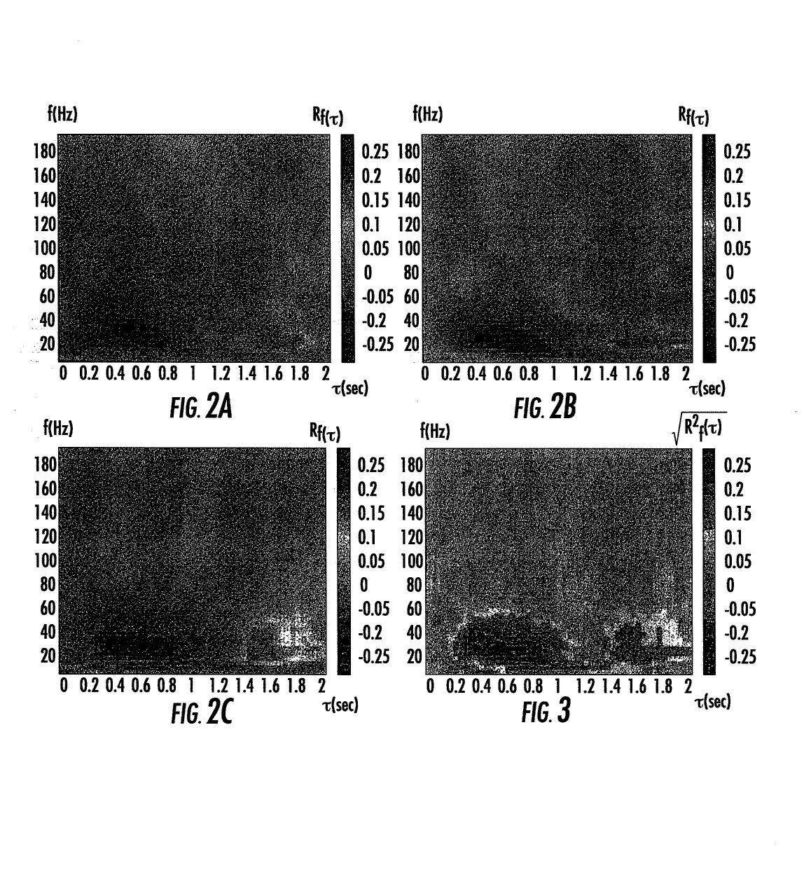

FIGS. 2A, 2B and 2C, three two-dimensional maps of the correlation coefficients between the signals acquired by three individual magnetoencephalographic sensors and a variable indicative of a visual stimulus;

FIG. 3, a two-dimensional map of a generalized determination coefficient combining the information contained in the maps of FIGS. 2B and 2C;

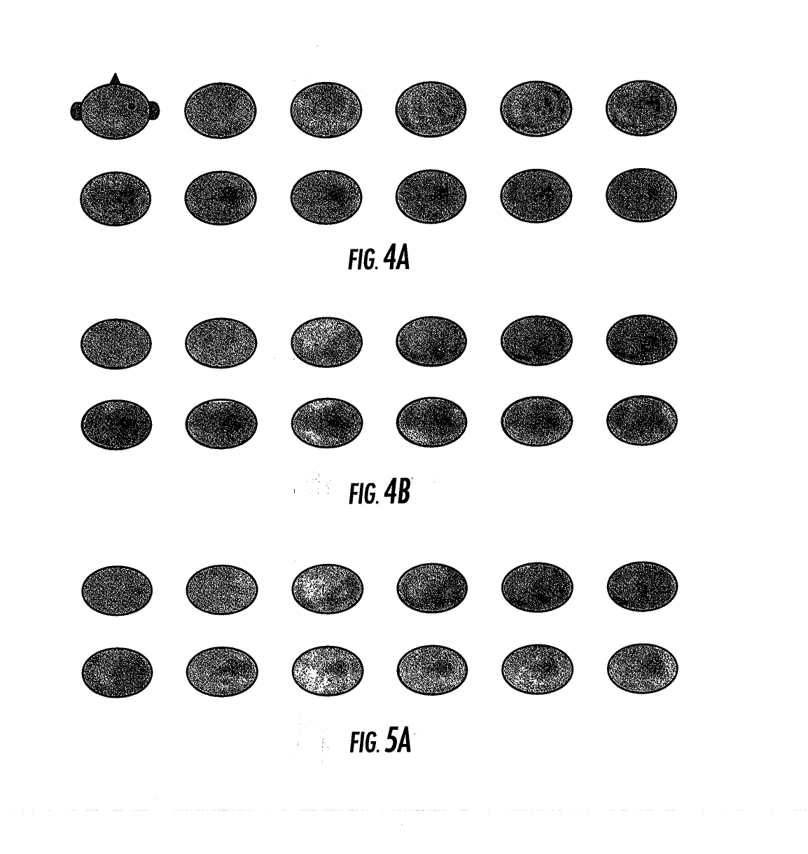

FIGS. 4A and 4B, maps of location of a brain activity obtained by a method equivalent to the prior art and by a method according to an embodiment of the invention, respectively;

FIGS. 5A, 5B and 5C, maps of location of a brain activity obtained by methods according to another embodiment of the invention; and

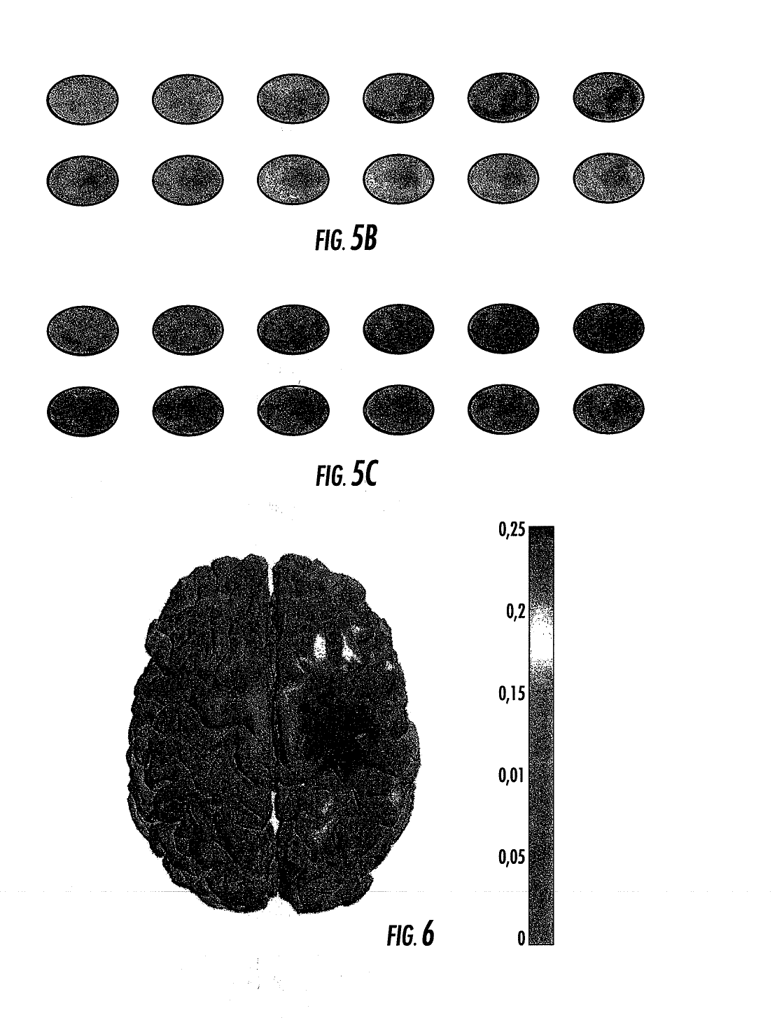

FIG. 6, a map representing an interpolation of the square root of determination coefficients, projected onto a three-dimensional representation of the cortical surface.

The MEG signals are generally acquired in a chamber provided with a magnetic shielding, by means of an “MEG headset” placed on the cranium of the subject, at a distance of 3-5 cm from the surface of the brain. The headset of FIG. 1 is made up of 102 sensors, or “MEG plates”, each comprising three individual sensors: a magnetometer and two first order planar gradiometers having sensitivity axes that are orthogonal and motionless in the plane of the plate, for a total of 306 individual sensors. These sensors make it possible to characterize the electrical currents of tangential orientation generated in the sulci of the brain. There are also other types of MEG sensors, such as radial gradiometers, but their use is less commonplace. For more information on this subject, reference can be made to the paper by J. Vrba and S. E. Robinson “Signal Processing in Magnetoencephalography”, Methods 25, 249-271 (2001).

The movements of the head of the subject are recorded and compensated by means of coils placed at predefined cardinal points (nasion, ear channels) and generating alternating magnetic fields at a frequency far from that of the MEG signals of interest.

In an experimental implementation of the method of the invention, the subject received the instruction to perform or imagine a movement of flexing/extending the left index finger in response to a visual stimulus. The signals acquired by the 306 individual sensors of the headset of FIG. 1 were sampled at a frequency of 1000 Hz and filtered to retain only the 0-200 Hz band. Then, they were subjected to a time-frequency decomposition by transformation into continuous wavelets. Thus, each signal sⁱ(t)(i=1-306 being the index making it possible to identify the individual sensor from which the signal is obtained) is represented by a time-frequency map xⁱ_{f}=CWT_{f}[sⁱ(t)], where CWT is the continuous wavelet transformation operator and “f” is the frequency.

The wavelet transformation can be followed by a smoothing and normalizing process. For example, it is possible to determine the absolute value of CWT_{f}[sⁱ(t)], then apply a sliding average, for example according to a duration of 300 ms, which can be expressed: Liss(abs(CWT_{f}[sⁱ(t)])), where “Liss” is the smoothing operator and “abs” the absolute value operator. Each frequency component of this quantity can then be normalized for example by its variance, which makes it possible to establish a balance between the values at low frequency, generally more intense than the values at high frequency.

It is possible to compute a Pearson correlation coefficient between each frequency component xⁱ_{f}(t) of an individual signal sⁱ(t) and a binary variable y(t) indicative of a visual stimulus. For example, y(t) can take the value 1 if the stimulus is emitted at the time t, and 0 otherwise. The Pearson correlation coefficient offset to the frequency f, R_{f}, is given by

$${R}_{f} ( τ ) = \frac{\frac{1}{n} \sum {x}_{f} ( t ) ( y ( t - τ ) - \overline{y} )}{{σ}_{{x}_{f}} {σ}_{y}} \qquad [ 1 ]$$

where:
y̅ is the average value of y(t) (it is assumed that xⁱ_{f}(t) is centered, that is to say that x̅_̅{̅f̅}̅=0, the generalization to the case of a non-centered variable is commonplace);
σₓ and σ_{y} are the variances of xⁱ_{f}(t) and y(t) respectively;
τ is the elapsed time since the stimulus, for example between 0 and 2 seconds, which means that interest is focused on the response from the brain within the 2 seconds which follow the emission of a stimulus;
n is the number of time samples.

FIGS. 2A-2C show graphs of R_{f}(τ) as a function of the frequency f and of the delay τ for three individual sensors (a magnetometer—FIG. 2A—and two orthogonal gradiometers—FIGS. 2B and 2C), identified by i=120, 121, and 122 respectively, borne by one and the same MEG sensor. These graphs show that the correlation coefficient takes a value substantially different from zero for the spectral components within the 10-40 Hz band, and that the coefficient is negative for τ between 0.2 and 1 sec., and positive for τ between 1.4 and 2 sec; the maximum value of |R_{f}(τ)| for the magnetometer 120 and the gradiometers 121 and 122 is respectively 0.17, 0.19 and 0.18 (the maximum possible value being 1). This indicates that it would be possible, for example, to produce a direct neural command by using the spectral components contained between 20 and 30 Hz of the signal s¹²¹(t) within the interval 0.4-0.8 seconds after the emission of a stimulus. By comparing between them the graphs associated with different MEG sensors, furthermore, it is possible to select those best suited for a BCI-type application. Since each sensor is sensitive to the electrical currents that circulate in the regions of the brain immediately adjacent, these graphs also make it possible to locate the brain activity of the subject in response to the stimulus.

More than the algebraic value of the coefficient R_{f}(τ), it is its absolute value which is of direct interest for the location of the brain activity and, consequently, for the determination of optimal places where sensors can be positioned for BCI applications. Consequently, it is possible to consider, instead of R_{f}(τ), its square R²_{f}(τ), called determination coefficient, or the square root thereof.

It can be shown that, in the condition (generally verified) that y(t) takes the value 0 most of the time—that is to say the condition that the stimuli are relatively short and spaced apart from one another—the information contained in the graphs of FIGS. 2A-2C is equivalent to that conveyed by the time-frequency maps, used in the abovementioned paper by Tadel et al. However, unlike the time-frequency maps, the determination coefficient R²_{f}(τ) can be generalized to the case of a vector variant. For example, it is possible to consider the variable x_{f}(t)=[x¹_{f}(t) . . . x^{N}_{f}(t)]^{T} (the exponent “^{T}” indicates the transposition operation), of which each element is a spectral component of an individual sensor (therefore N=2 if the signals from 2 gradiometers are considered).

In practice, the determination coefficient estimates the fraction of the variance of y(t) which is explained by x_{f}(t) by means of a (multi)linear regression:

$$\begin{matrix}{R}_{f}^{2} ( τ ) = 1 - \frac{\sum {( y ( t ) - \hat{y} ( t ) )}^{2}}{\sum {( y ( t ) - \overline{y} )}^{2}} & [ 2 ] \\ with \hat{y} ( t ) = a + {{\sum }_{i = 1}}^{N} {b}_{i} {x}_{f}^{i} ( t + τ ) & [ 3 ]\end{matrix}$$

the coefficients a and bᵢ of the equation [3] being calculated by the least squares method.

Generally, this determination coefficient expresses the correlation between y(t) and the estimation of y(t), denoted ŷ(t), using the variable x_{f}(t+τ).

FIG. 3 is a two-dimensional graph of \sqrt{R²_{f}(τ)} obtained by considering the variable x_{f}(t+τ)=[x¹²¹_{f}(t+τ), x¹²²_{f}(t+τ)]^{T} which combines the signals from the two gradiometers 121, 122.

More specifically, from this variable, it is possible to construct a matrix

$$X = ( \begin{matrix}1 & {x}_{f}^{1} ( {t}_{1} + τ ) & {x}_{f}^{2} ( {t}_{1} + τ ) \\ 1 & {x}_{f}^{1} ( {t}_{2} + τ ) & {x}_{f}^{2} ( {t}_{2} + τ ) \\ … & … & … \\ 1 & {x}_{f}^{1} ( {t}_{n} + τ ) & {x}_{f}^{2} ( {t}_{n} + τ )\end{matrix} ) ,$$

x¹_{f}(t₁+τ; representing the spectral component in the frequency band f of the gradiometer i measured at the instant t+τ following the instant tⱼ of sampling of the variable y(t). The time offset, denoted τ, is generally greater than 0 s and less than 5 s, preferably less than 2 s, for all the embodiments.

A vector y is also constructed that combines the different values y(tⱼ) such that y=(y(t₁) y(t₂) . . . (y(t_{N}))^{T}.

ŷ(t)=b₀+b₁x¹f(t+τ)+b₂x_{f}²(t+τ), is then determined, b₀, b₁ and b₂ being estimated by the least squares method, for example according to

b=(X^{T}X)⁻¹X^{T}y, with b=(b₀ b₁ b₂)^{T}

The determination coefficient is then calculated by applying the equation [2].

Thus, according to this embodiment,
a number N of signals is used, N here being equal to 2, the signals being measured at one and the same point (or at least in one and the same region of the brain within the meaning of the invention).
the frequency component of each signal is determined at the frequency f, which makes it possible to construct a variable x_{f}(t+τ), each term of the variable x_{f}(t+τ) comprising the frequency component, at the frequency f, denoted x^{k}_{f}(t+τ) of a signal x^{k} measured by the gradiometer k at an instant t+τ. The dimension of this variable x_{f}(t+τ) is N.
A correlation is established between a variable representative of an action imagined or performed by a subject (in this case, y(t)) and the variable x_{f}(t+τ) with N dimensions.

It will be noted that, in this example, each component of the variable x_{f}(t+τ) is determined using just one of the N signals measured for said region of the brain. Moreover, the variable T represents the time offset between the signals x^{k} considered and the estimation of the variable y(t).

The maximum value of \sqrt{R²_{f}(τ)} is 0.21, to be compared to a value of 0.19 obtained by taking into consideration a single gradiometer. Thus, by combining different signals, recorded at the same place, it is possible to increase the sensitivity of the detection of a modification of the brain activity in response to a stimulus.

FIGS. 4A and 4B show spatial maps of the brain activity (imagined movement of the left index finger) of a subject in response to a visual stimulus, at different instants after the emission of said stimulus. More precisely:
the maps of FIG. 4A show the correlation coefficients R(τ) between the signals obtained from a gradiometer for each MEG sensor and the binary variable y(t); the delay τ is between 0.00 sec, for the left-hand map of the first line, and 0.88 sec for the right-hand map of the second line, with increments of 0.08 sec between each image.

This figure shows, for a gradiometer of each sensor, the value of the correlation coefficient that has the highest absolute value, whether the coefficient is positive or negative. In other words, this figure represents

$${extremum}_{f} [ {R}_{f} ( τ ) ] ,$$

in which “extremum” is the operator which associates with the function which constitutes the argument thereof its extreme value (maximum or minimum), which, in practice is equivalent to

$${max}_{f} \sqrt{{R}_{f}^{2} ( τ )} ,$$

the negative correlations generally having a low absolute value.

The circle on the first map of the first line shows the location of the individual sensors 120-122 mentioned above.
the maps of FIG. 4B show the square roots of the maximum determination coeffients

$${max}_{f} \sqrt{{R}_{f}^{2} ( τ )}$$

calculated by taking into consideration the signals obtained from the two gradiometers of each MEG sensor. As for FIG. 4A, the delay τ is between 0.00 sec and 0.68 sec with increments of 0.08 sec between each image, and the coefficients are integrated between 5 and 200 Hz.

It can be noted that the multichannel analysis (FIG. 4B) allows for a better location of the brain activity. From a quantitative point of view this is expressed by the fact that, in the case of the single channel analysis of FIG. 4A, |R(τ)| takes a maximum value of 0.2 whereas, in the case of FIG. 4B, the maximum value of \sqrt{R²(τ)} reaches 0.3.

The use of the generalized determination coefficients also allows for a better use of the frequency information. Thus, it is possible to use a variable x(t)=[x¹_{f1}(t) . . . x¹_{fM}(t) . . . x^{N}_{f1}(t) . . . X^{Nc}_{fM}(t)]^{T}. This variable combines the signals from N individual sensors by taking into consideration M spectral components (indices f1-fM) for each of them. Its dimension is N·M.

More specifically if tⱼ represents the instant at which the value of y(t) is acquired, the variable x (tᵢ+τ)=[x¹_{f1}(tᵢ+τ) . . . x¹_{fM}(tᵢ+τ) . . . x^{Nc}_{f1}(tᵢ+τ) . . . X^{Nc}_{fM}(tᵢ+τ)]^{T}, is used, τ being the time interval separating the measurement from this instant tᵢ.

It is possible to establish, at each interval τ, a regression coefficient R(τ) by constructing the matrix X, of which each line comprises the variable x (tᵢ+τ):

$$X = ( \begin{matrix}1 & {x}_{f 1}^{1} ( {t}_{1} + τ ) & {x}_{f 2}^{1} ( {t}_{1} + τ ) & … & {x}_{f 1}^{2} ( {t}_{1} + τ ) & {x}_{f 2}^{2} ( {t}_{1} + τ ) & … \\ 1 & {x}_{f 1}^{1} ( {t}_{2} + τ ) & {x}_{f 2}^{1} ( {t}_{2} + τ ) & … & {x}_{f 1}^{2} ( {t}_{2} + τ ) & {x}_{f 2}^{2} ( {t}_{2} + τ ) & … \\ … & … & … & … & … & … & … \\ 1 & {x}_{f 1}^{1} ( {t}_{N} + τ ) & {x}_{f 2}^{1} ( {t}_{N} + τ ) & … & {x}_{f 1}^{2} ( {t}_{N} + τ ) & {x}_{f 2}^{2} ( {t}_{n} + τ ) & …\end{matrix} )$$

and the observations vector y=(y(t₁) y(t₂) . . . y(t_{N}))^{T}.

In the expression of the matrix X, xⁱ_{f}_{ₖ}(tⱼ+τ) represents the spectral component in the frequency band fₖ of the gradiometer i measured at the instant t+τ following the instant tⱼ of sampling of the variable y(t).

The regression coefficient R(τ) is obtained by determining

$$\hat{y} ( t ) = {b}_{0} + {\sum }_{i = 1}^{M} {b}_{i}^{1} {x}_{fi }^{1} ( t + τ ) + {\sum }_{i = 1}^{M} {b}_{i}^{2} {x}_{fi}^{2} ( t + τ )$$

and:

b=(b₀ b₁¹ b₂¹ . . . b₁² b₂² . . . b_{M}²)^{T} such that b=(X^{T}X)⁻¹X_{y}.

Thus, according to this embodiment,
a number N of signals is used, N here being equal to 2, the signals being measured at one and the same point (or in one and the same region of the brain within the meaning of the invention)
the frequency component of each signal is determined at different frequencies f₁ . . . f_{M}, which makes it possible to construct a variable x (t+τ)=[x¹_{f1}(t+τ) . . . X¹_{fM}(t+τ) . . . X^{N}_{f1}(t+τ) . . . X^{N}_{fM}(t+τ)]^{T}, in which each term of the variable x(t+τ) comprises the frequency component, at the frequency fᵢ, denoted x^{k}_{fi}(t+τ) of a signal x^{k} measured at an instant t+τ by the sensor k. The dimension of this variable x^{k}_{f}(t+τ) is N*M, N being the number of signals addressing the same region of the brain and M being the number of frequency components,
a correlation is established between a variable representative of an action imagined or performed by a subject (in this case y(t)) and the variable x(t+τ) with N dimensions.

It will be noted that, in this example, each component of the variable x(t+τ) is determined using just one of the N signals measured for said region of the brain. Moreover, the variable τ represents the time offset between the signals x^{k} considered and the variable y(t).

FIGS. 5A-5C show brain activity maps obtained in conditions similar to those in FIG. 4B, by combining one or more spectral components of signals obtained from two gradiometers for each MEG sensor. However:
in the case of FIG. 5A, a single spectral component in the region 5-12 Hz was considered: Nc=2, M=1, N=2. The maximum value of \sqrt{R²(τ)} comes to 0.14.
in the case of FIG. 5B, three spectral components in the region 5-12 Hz were considered: Nc=2, M=3, N=6. The maximum value of \sqrt{R²(τ)} comes to 0.2
in the case of FIG. 5C, 21 spectral components in the region 5-200 Hz were considered: Nc=2, M=21, N=42. The maximum value of \sqrt{R²(τ)} comes to 0.35.

The maximum value of \sqrt{R²(τ)} increases with the number M of spectral components considered for the analysis.

However, in the case of FIG. 5C, a decrease in the contrast is observed which hampers the location of the brain activity. This is a problem of overlearning, which can be remedied by known techniques.

One possible technique is to apply a cross-validation algorithm, by dividing all of the signals measured into Q subsets. Q sub-matrices X_{q} are then constructed, each corresponding to a vector y_{q}.

Each term of a subvector ŷ_{q} is then estimated according to the preceding relationship, the vector b^{q}=(b₀^{q} b₁^{1q} b₂^{1q} . . . b₁^{2q} b₂^{2q} . . . b_{M}^{2q})^{T} being determined by using the sub-matrices Xₚ, with p≠q.

Furthermore, a Ridge regression algorithm can be implemented which allows for a more stable determination of the vector b.

A new series of tests was performed. The table below represents a comparison of the maximum values of the square root of the determination coefficient \sqrt{R²(τ)}, either for all the sensors, or for all the frequencies and all the sensors, this coefficient being obtained by performing different actions during the observation of a stimulus:

1st line: imaginary movement of the left index finger

2nd line: actual movement of the left index finger

3rd line: imaginary movement of the right index finger

4th line: actual movement of the right index finger.

The first column of the table indicates the maximum values, for all the frequencies considered, of the square root of the determination coefficient, by using values obtained from a single gradiometer, that is to say

$${max}_{f , τ} \sqrt{{( {R}_{n , f} ( τ ) )}^{2}} ,$$

where R_{n,f}(τ) represents the correlation coefficient obtained by using the spectral component at the frequency f measured by the gradiometer n at the instant τ.

The second column of the table indicates the maximum values of the square root of the determination coefficient, for all the frequencies considered, by using the modulus of the sum of the signals produced at one and the same point, by two gradiometers, that is to say

$${max}_{f , τ} \sqrt{{( {R}_{f} ( τ ) )}^{2}} ,$$

where R_{f}(τ) represents the correlation coefficient obtained by using the spectral component at the frequency f of the modulus of the sum of the signals produced by two orthogonal gradiometers situated at the same point n at the instant τ.

The third column of the table indicates the maximum values of the square root of the determination coefficient, by combining the spectral components at one and the same point, of the signals measured by two gradiometers

$${max}_{τ} \sqrt{{( {R}_{f} ( τ ) )}^{2}} ,$$

where R(τ) represents the correlation coefficient obtained by using the different spectral components of the signals produced by two orthogonal gradiometers situated at the same point n at the instant τ. The results represented in this column were obtained by combining the signals from 2 gradiometers in the 21 frequency bands considered.

| | ${}_{f , τ}^{max} \sqrt{{( {R}_{n , f} ( τ ) )}^{2}}$ | ${}_{f , τ}^{max} \sqrt{{( {R}_{f} ( τ ) )}^{2}}$ | ${}_{τ}^{max} \sqrt{( {{R}_{f} ( τ )}^{2}}$ |
|---|---|---|---|
| Left index | 0.192 | 0.206 | 0.3 |
| finger | | | |
| (imaginary) | | | |
| Left index | 0.252 | 0.363 | 0.461 |
| finger | | | |
| (actual) | | | |
| Right index | 0.234 | 0.228 | 0.292 |
| finger | | | |
| (imaginary) | | | |
| Right index | 0.375 | 0.374 | 0.470 |
| finger | | | |
| (actual) | | | |

These experimental results show that a higher determination coefficient is obtained when different spectral components of the signals obtained from a plurality of gradiometers (in this case two) positioned at one and the same point (3rd column) are combined.

According to the different embodiments described above, the end result obtained is one or more correlation coefficients for each measurement point as a function of the duration T between the stimulus and the measurement. Correlation (or determination) coefficient values are then available according to a spatial mesh defined by the positioning of the sensors. This mesh can be used as a basis for projecting said values onto the surface of the cortex. For this, the surface of the cortex is obtained, for example from MRI measurements, and then is modeled. The mesh formed by the different sensors is then readjusted in relation to this model, for example, by using stereotaxic markers that can be seen in MRI imaging, notably gadolinium salt pellets positioned on the head of the patient.

From the determination coefficient values, a projection is performed onto the model of the cortical surface, the value assigned to each element of said cortical surface being derived from an interpolation between different points of the mesh, for example the three closest neighbors (S, S1, S2), the weighting criterion being a distance. An image is thus obtained as in FIG. 6. This figure was obtained by interpolating the square root values of the determination coefficient at 0.48 s of a stimulus, on seeing which the patient must imagine the movement of the left index finger.

The invention has been described with reference to particular embodiments, using MEG sensors comprising magnetometers and first order planar gradiometers and a binary variable y representative of a visual stimulus. However, these are in no way essential limitations. In fact, the method of the invention can be applied to different sensors; it also allows signals obtained from sensors of different kinds—for example magnetometers and EEG or ECoG electrodes—to be combined. Moreover, the variable y is not necessarily binary, it can more generally, take discrete or continuous values. Furthermore, the correlation between the multidimensional variable x and the scalar variable y can be quantified otherwise than by a determination coefficient.

1. Method for locating a brain activity, comprising the steps:
a) acquiring data indicative of sensory stimuli addressed to, or deliberate actions performed or imagined by, a subject;
b) by means of a plurality of sensors, acquiring signals representative of an activity, associated with said stimuli or deliberate actions, of respective regions of the brain of said subject; and
c) for each said sensor, quantifying a correlation that exists between said data indicative of sensory stimuli or of deliberate actions and the signals acquired;
characterized in that N≧2 signals are acquired that are representative of N distinct physical quantities, measured for one and the same region of the brain, and in that said correlation is established between a variable (y) indicative of a said sensory stimulus or of a said deliberate action and a variable (x_{f}, x) of at least N dimensions representative of said signals.

2. Method according to claim 1, in which said correlation is established between said variable indicative of a said sensory stimulus or of a said deliberate action at the instant t (y(t)) and said variable of at least N dimensions (x_{f}(t+τ), x(t+τ)), representative of said signals measured for one and the same region of the brain, said signals being offset in time relative to said instant t.

3. Method according to claim 1, in which said correlation that exists between said data indicative of sensory stimuli or of deliberate actions and the signals acquired by each said sensor is quantified by means of a determination coefficient.

4. Method according to claim 1, in which said sensors are magnetoencephalographic sensors.

5. Method according to claim 3, in which each said magnetoencephalographic sensor acquires at least one signal representative of a magnetic field intensity and one signal representative of a component of a gradient of said magnetic field.

6. Method according to claim 3, in which each said magnetoencephalographic sensor acquires at least two signals representative of two components of a gradient of said magnetic field.

7. Method according to claim 1, in which said variable of at least N dimensions is obtained by selecting at least one spectral component of each of said signals, acquired over a time window associated with a said sensory stimulus or deliberate action.

8. Method according to claim 1, in which said multidimensional variable is obtained by selecting a plurality of spectral components of a signal acquired over a time window associated with a said sensory stimulus or deliberate action.

9. Method according to claim 1, also comprising a visualization step in which values indicative of said correlation, determined for each said sensor, are projected onto a three-dimensional model of a cortical surface, and an interpolation of said values between different points of a mesh of said surface is performed.

10. Method for locating brain activity sensors for direct neural control comprising:
a step of locating a brain activity, implemented by a method according to claim 1; and
a step of determining optimum locations of said brain activity sensors according to the results of said step of locating a brain activity.
